(19) United States
(12) Patent Application Publication
(10) Pub. No.: US 2022/0293211 A1
(43) Pub. Date: Sep. 15, 2022

(54) Automated Interpretation of Protein Capillary Electrophoresis Data

(71) Applicants: Andrew Hughes, St. Louis, MO (US); Ann Gronowski, St. Louis, MO (US); Christopher Farnsworth, St. Louis, MO (US)

(72) Inventors: Andrew Hughes, St. Louis, MO (US); Ann Gronowski, St. Louis, MO (US); Christopher Farnsworth, St. Louis, MO (US)

(73) Assignee: Washington University, St. Louis, MO (US)

(21) Appl. No.: 17/694,679

(22) Filed: Mar. 15, 2022

Related U.S. Application Data
(60) Provisional application No. 63/160,486, filed on Mar. 12, 2021.

(51) Int. Cl. G16B 15/20; G16B 40/00

(52) U.S. Cl. CPC G16B 15/20; G16B 40/00

(57) ABSTRACT

Serum protein electrophoresis (SPEP) analysis systems and methods for automatically generating appropriate clinical interpretations of SPEP data are disclosed.

### CROSS-REFERENCE TO RELATED APPLICATIONS

This application claims priority from U.S. Provisional Application Ser. No. 63/160,486 filed on 12 Mar. 2021, the content of which is incorporated herein by reference in its entirety.

### STATEMENT REGARDING FEDERALLY SPONSORED RESEARCH OR DEVELOPMENT

Not applicable.

### MATERIAL INCORPORATED-BY-REFERENCE

Not applicable.

### FIELD OF THE DISCLOSURE

The present disclosure generally relates to serum protein electrophoresis (SPEP) analysis systems and methods, and in particular, the present disclosure relates to SPEP analysis systems and methods for automatically generating appropriate clinical interpretations of SPEP data.

Other objects and features will be in part apparent and in part pointed out hereinafter.

### BACKGROUND OF THE DISCLOSURE

Protein capillary electrophoresis is an analytical method that separates proteins based on size and charge, and it is widely used to characterize patient specimens of interest. The two most common clinical applications are serum protein electrophoresis (used to detect and monitor clonal antibodies associated with B cell disorders) and hemoglobin electrophoresis (used to diagnose and monitor hemoglobinopathies). The data generated by protein capillary electrophoresis are two-dimensional curves, and interpreting these requires manual review by a specialist trained to identify a range of normal and abnormal patterns. Protein capillary electrophoresis is used to detect and monitor clonal immunoglobulins associated with multiple myeloma and other clonal plasma cell disorders.

Existing workflows for protein capillary electrophoresis analysis entail manually reviewing results for each specimen to determine appropriate diagnostic comments. The manual review process is subjective, time-consuming, requires specialty training, and is susceptible to transcriptional errors as well as inconsistency across reviewers. Given the high number of serum protein electrophoresis tests performed at hospitals and other treatment facilities, augmenting the methods used in existing workflows with accurate, automatically-generated interpretative comments would save several hours of hands-on time per week, decrease turnaround time, reduce the training needed by technologists, standardize the results reported to clinicians, and decrease transcriptional errors.

### DESCRIPTION OF THE DRAWINGS

In various aspects, a computer-implemented method for automatically generating diagnostic comments for protein capillary electrophoresis data obtained for a subject is disclosed that includes providing at least one two-dimensional serum protein electrophoresis (SPEP) profile comprising a plurality of measured abundances and corresponding times; extracting, using the computing device, a feature set from the SPEP profile, the feature vector comprising at least one feature of the at least one two-dimensional protein electrophoresis profile, wherein the at least one feature comprises at least one identified peak, at least one region corresponding to each identified beak, at least one peak feature associated with each identified peak, and at least one region feature associated with each region; and transforming, using a machine-learning model implemented on the computing device, the feature vector into the diagnostic comments and corresponding confidences of each diagnostic comment. In some aspects, the peak feature comprises at least one of an x-coordinate, a y-coordinate, a local curvature (3-unit window), a local angle (3-unit window), a leading and a lagging first derivative (mean, 5-unit window), a leading and a lagging second derivative (mean, 5-unit window), and any combination thereof. In some aspects, the at least one region feature comprises at least one of an area under the curve, a skew, a number of inflection points, a mean curvature, a minimum of the second derivative, a mean sum of squares of the second derivative, at least one slope of a segments connecting each region boundary to its associated peak, an angle formed by adjacent peaks through a joining boundary, at least one root mean squared errors of polynomial fit (degree 2, 4, 6, 8, and 10) and any combination thereof. In some aspects, extracting the feature set further comprises determining, using the computing device, a plurality of candidate peaks, selecting a portion of the candidate peaks with the lowest second derivatives. In some aspects, extracting the feature set further comprises assigning, using the computing device, each candidate peak of the portion to a corresponding reference peak, wherein each reference peak is a known serum protein selected from albumin, alpha-1, alpha-2, beta-1, beta-2, and gamma. In some aspects, assigning each candidate peak further comprises assigning one or two additional candidate peaks to secondary peaks comprising secondary beta-2 or secondary gamma. In some aspects, the machine learning model comprises one of KNN, elastic net regression, random forests, and gradient boosting machine.

Other aspects of the disclosure are disclosed herein.

### DESCRIPTION OF THE DRAWINGS

Those of skill in the art will understand that the drawings, described below, are for illustrative purposes only. The drawings are not intended to limit the scope of the present teachings in any way.

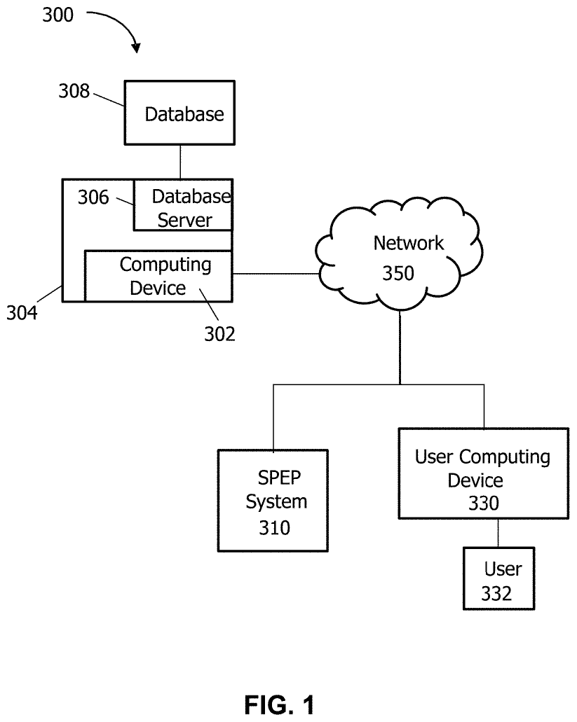

FIG. 1 is a block diagram schematically illustrating a system in accordance with one aspect of the disclosure.

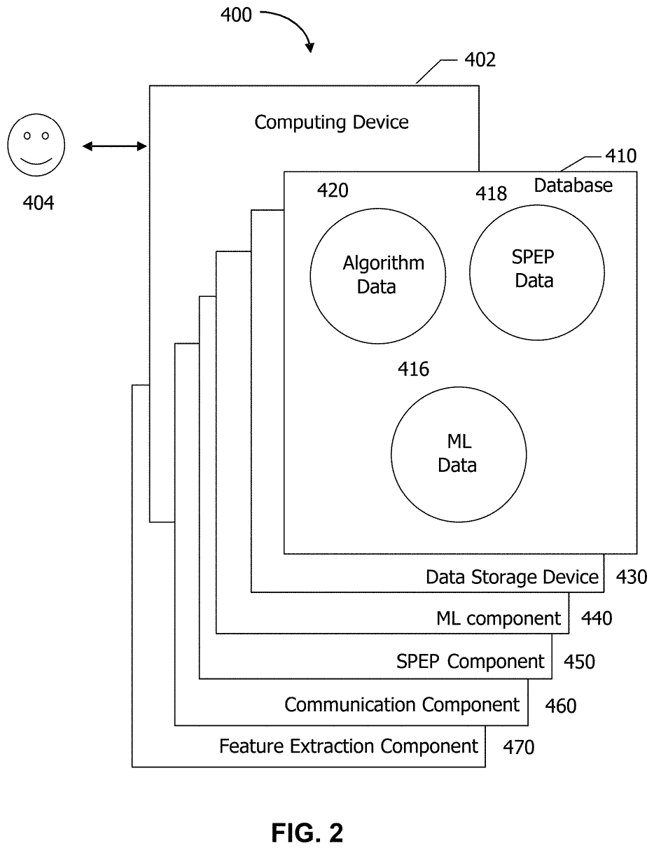

FIG. 2 is a block diagram schematically illustrating a computing device in accordance with one aspect of the disclosure.

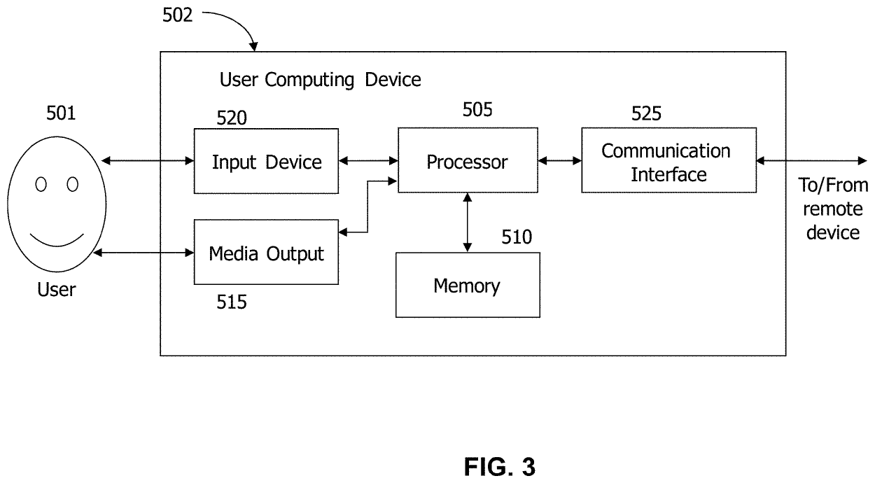

FIG. 3 is a block diagram schematically illustrating a remote or user computing device in accordance with one aspect of the disclosure.

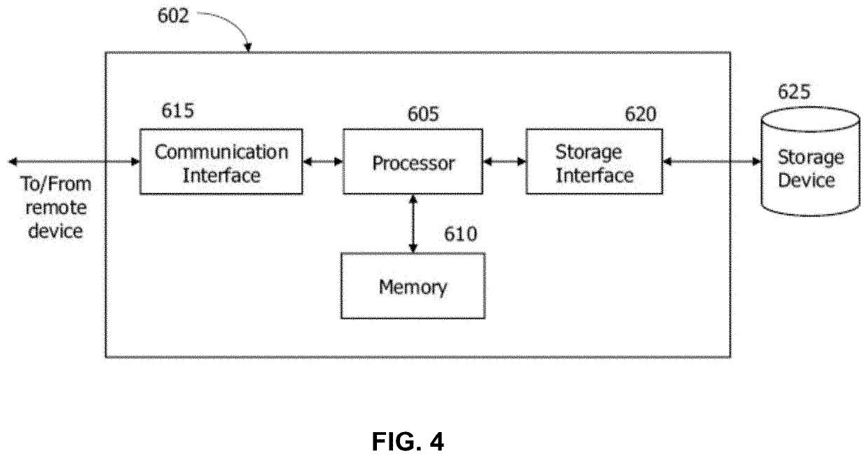

FIG. 4 is a block diagram schematically illustrating a server system in accordance with one aspect of the disclosure.

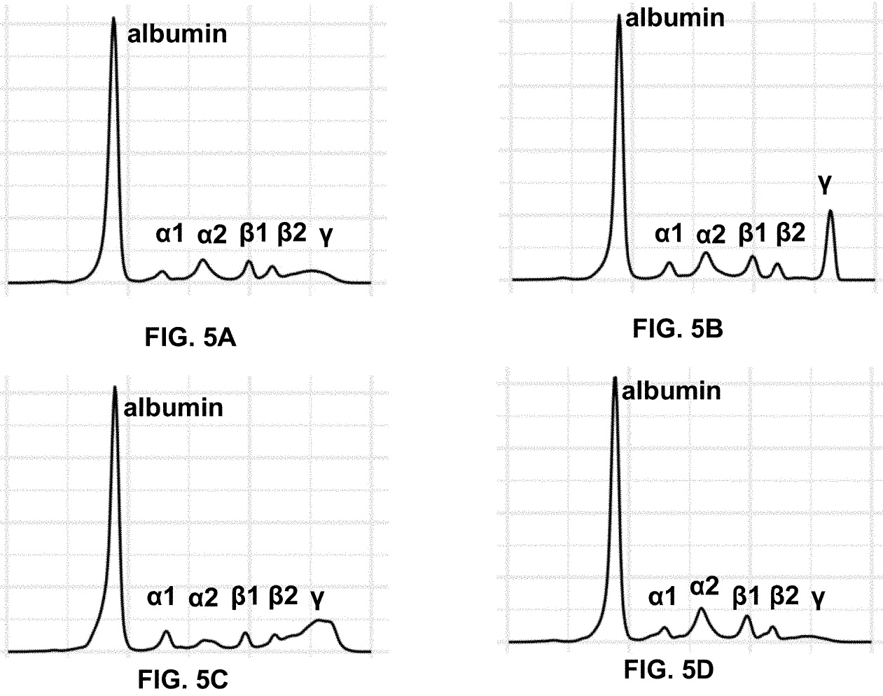

FIG. 5A is a graph illustrating an exemplary two-dimensional serum protein electrophoresis (SPEP) profile with a normal profile.

FIG. 5B is a graph illustrating an exemplary two-dimensional serum protein electrophoresis (SPEP) profile with an abnormal peak in the gamma region.

FIG. 5C is a graph illustrating an exemplary two-dimensional serum protein electrophoresis (SPEP) profile with an abnormal peak in the gamma region.

FIG. 5D is a graph illustrating an exemplary two-dimensional serum protein electrophoresis (SPEP) profile with a possible abnormal peak in the gamma region.

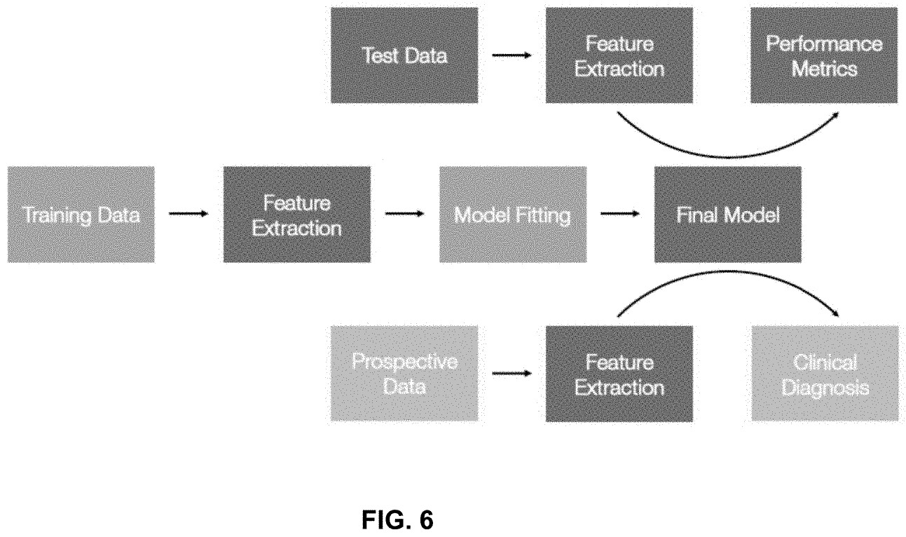

FIG. 6 is a flow chart illustrating a method of automated interpretation of protein capillary electrophoresis data in one aspect.

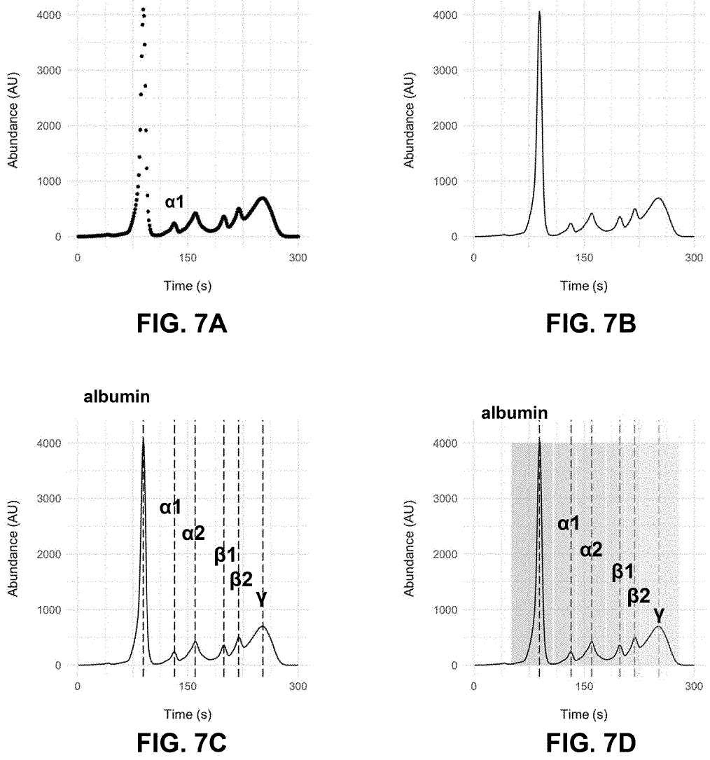

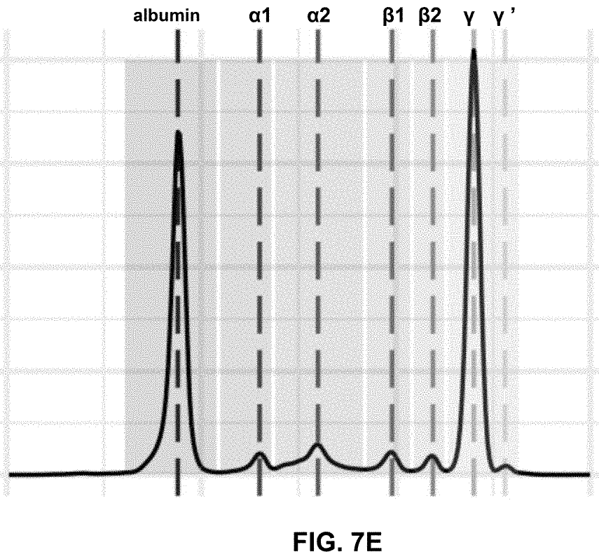

FIG. 7A is a graph illustrating an exemplary two-dimensional serum protein electrophoresis (SPEP) profile to be interpreted using the disclosed method of automated interpretation of protein capillary electrophoresis data in one aspect.

FIG. 7B is a graph of the SPEP profile data of FIG. 7A after smoothing.

FIG. 7C is a graph showing the smoothed SPEP profile of FIG. 7B with protein peaks identified using the disclosed method; peaks are denoted with vertical dashed lines.

FIG. 7D contains the graph of FIG. 7C segmented into protein peak regions (shaded) using the disclosed method.

FIG. 7E is a graph of a smoothed SPEP profile segmented into protein peak regions (shaded) that includes an additional/secondary protein peak denoted as γ′.

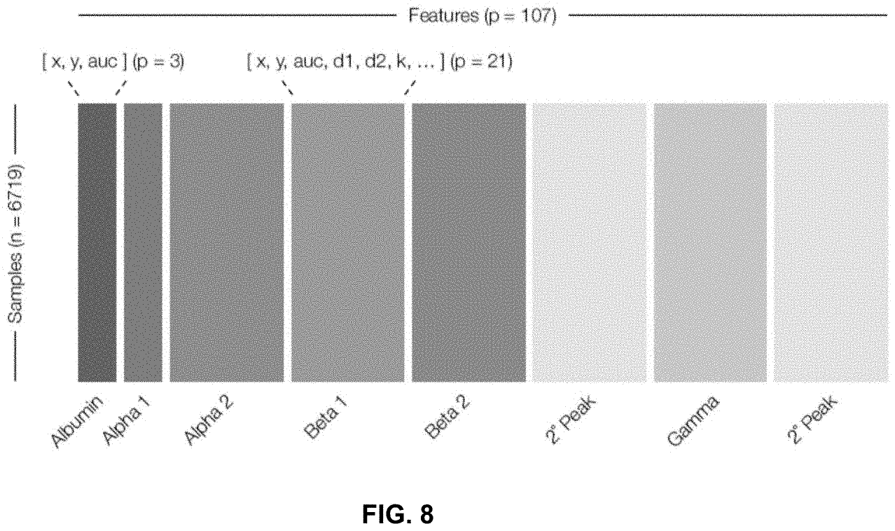

FIG. 8 is a diagram illustrating a features matrix in one aspect.

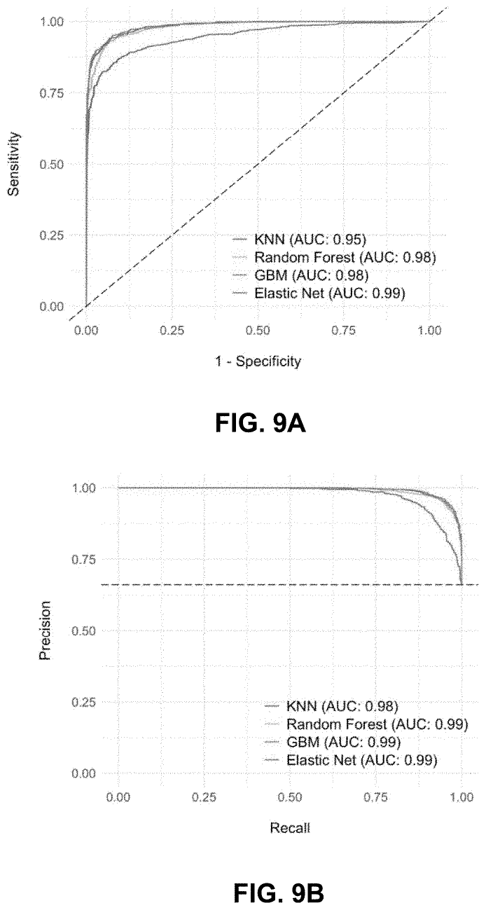

FIG. 9A is an ROC graph comparing the sensitivity/specificity performance of four different machine learning models with respect to interpreting protein capillary electrophoresis data.

FIG. 9B is a graph comparing the precision/recall performance of four different machine learning models with respect to interpreting protein capillary electrophoresis data.

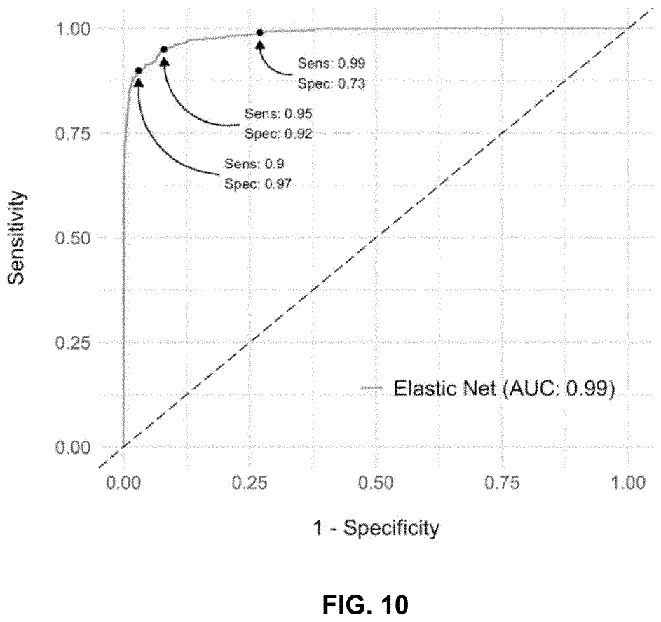

FIG. 10 is an ROC graph obtained using an elastic net regression model, with several operating points identified.

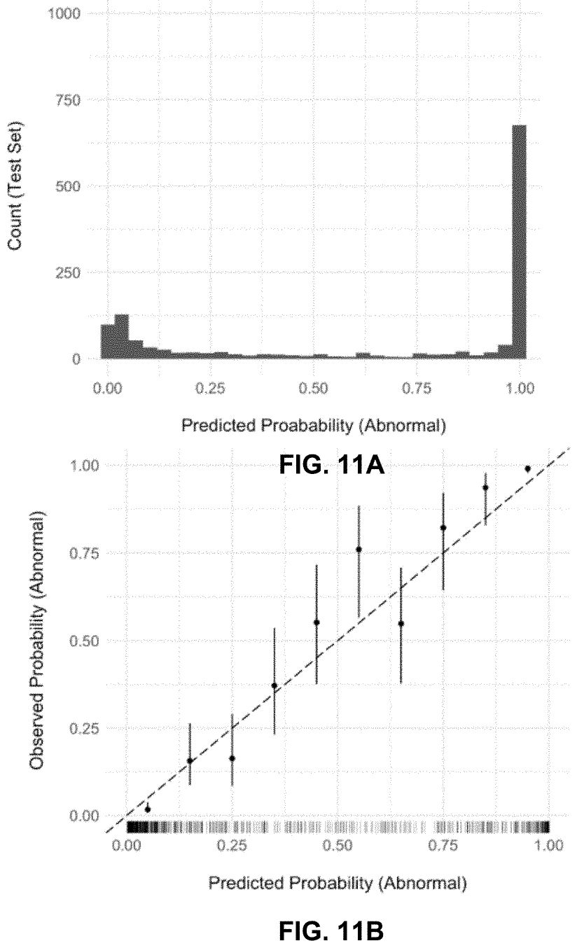

FIG. 11A is a histogram of the number of protein capillary electrophoresis test sets arranged by the probability of an abnormal 2D protein profile predicted by an elastic net regression model.

FIG. 11B is a graph of the observed probability of an abnormal 2D profile as a function of the corresponding probability of an abnormal 2D profile as predicted by an elastic net regression model.

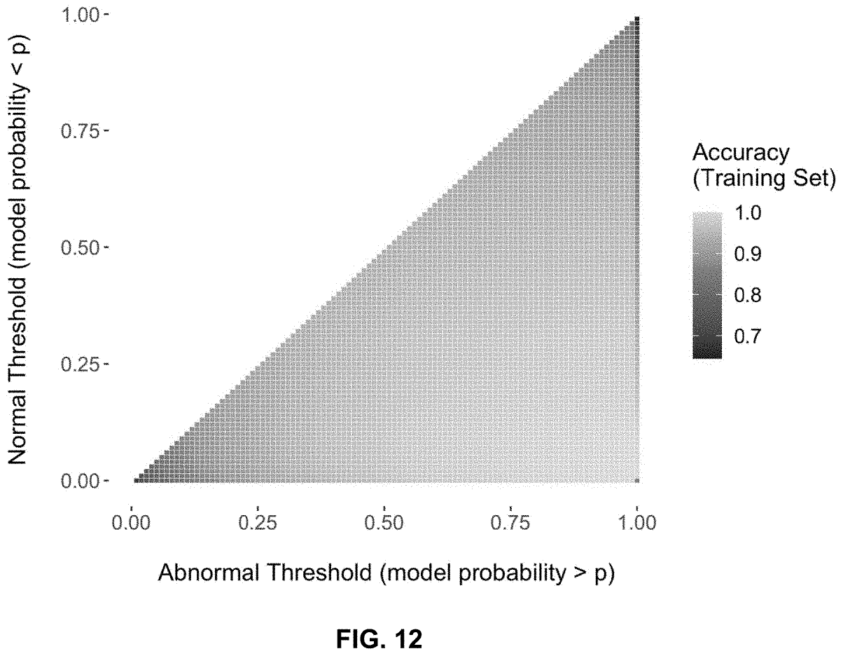

FIG. 12 is a graph summarizing the accuracy of predicted normal and abnormal 2D protein profile predictions as a function of probability thresholds.

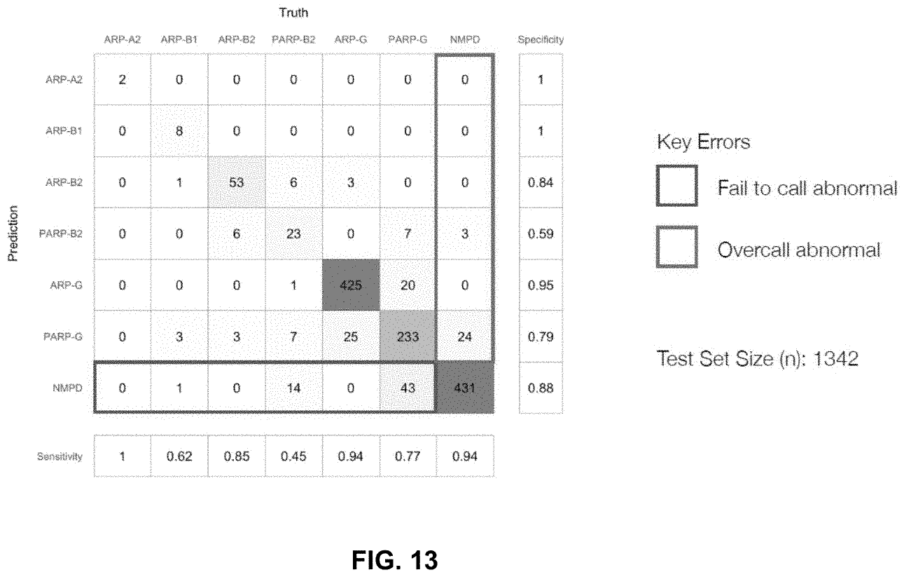

FIG. 13 is a truth table summarizing prediction errors using a GBM machine learning model.

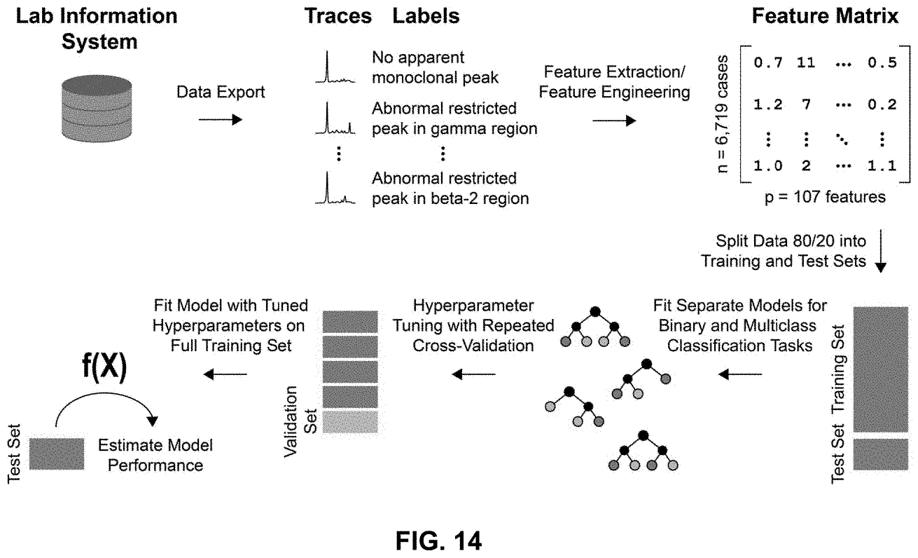

FIG. 14 is a schematic diagram illustrating the development and validation of various machine learning models for the automated analysis of SPEP profiles.

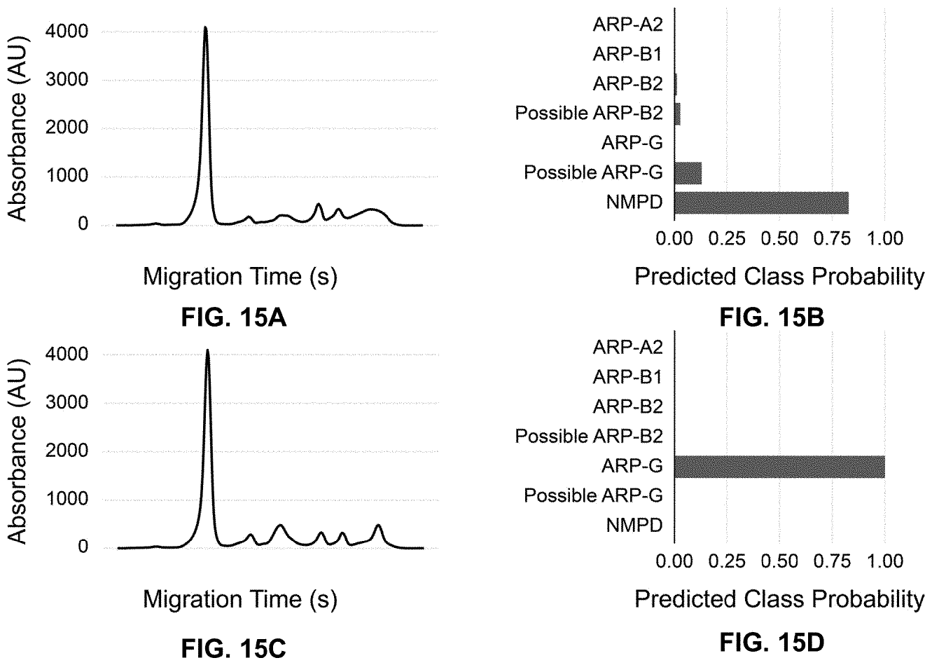

FIG. 15A is an example SPEP trace to be analyzed using an automated method in accordance with one aspect of the disclosure.

FIG. 15B is a bar graph summarizing the predicted class probabilities based on the SPEP trace of FIG. 15A in accordance with one aspect of the disclosed method.

FIG. 15C is an example SPEP trace to be analyzed using an automated method in accordance with one aspect of the disclosure.

FIG. 15D is a bar graph summarizing the predicted class probabilities based on the SPEP trace of FIG. 15C in accordance with one aspect of the disclosed method.

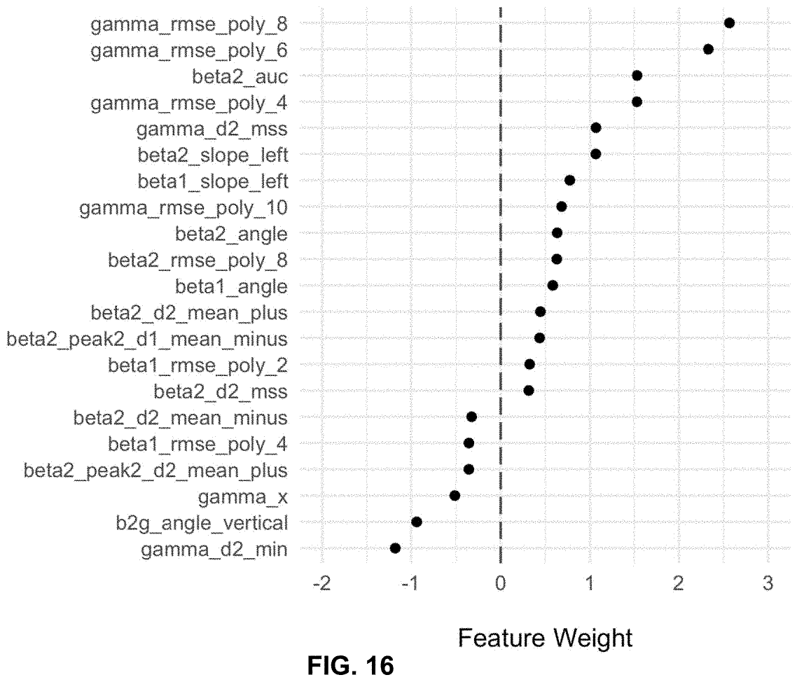

FIG. 16 is a graph summarizing the weighting of individual features within a feature set by a machine learning model used in the disclosed method of automated analysis of SPEP profiles in one aspect.

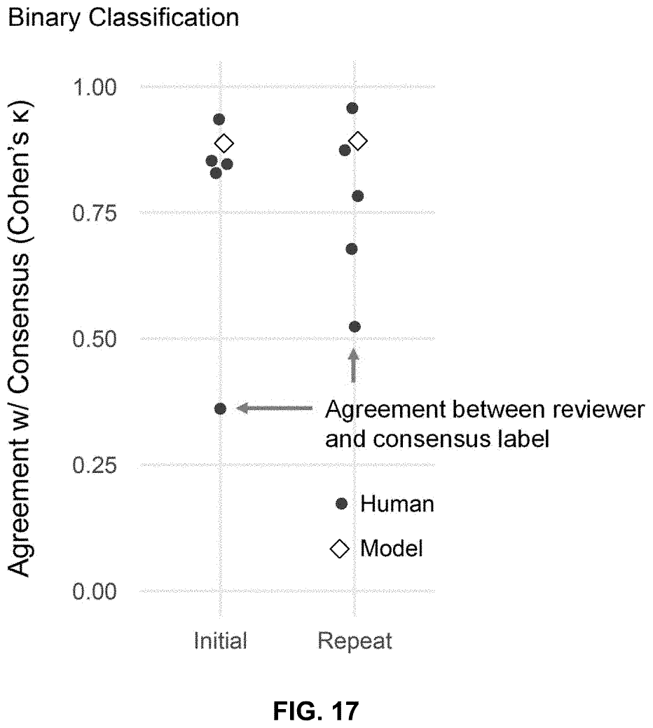

FIG. 17 is a graph summarizing the agreement with a consensus result of practitioner interpretation (circles) and machine learning model-derived (diamonds) binary classifications of SPEP traces for a single analysis and for repeated analyses.

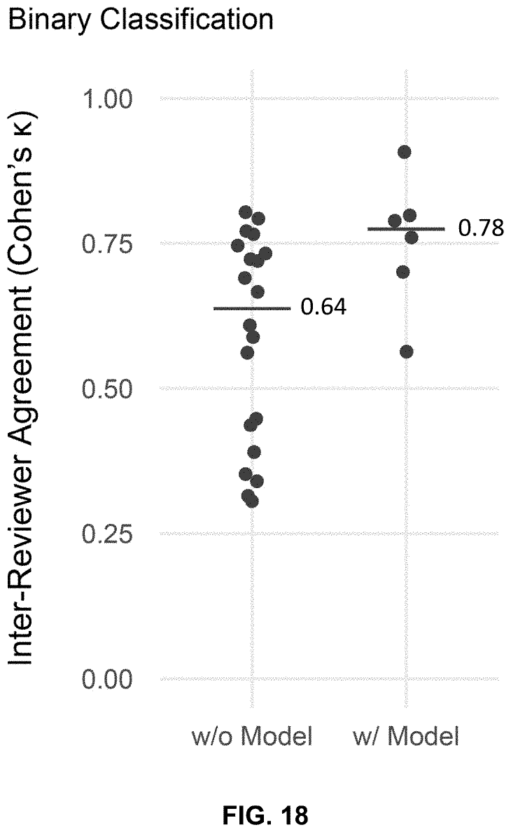

FIG. 18 is a graph summarizing the agreement between interpretations of the SPEP traces by the same practitioner with (right) and without knowledge of the corresponding classifications obtained using a machine learning model.

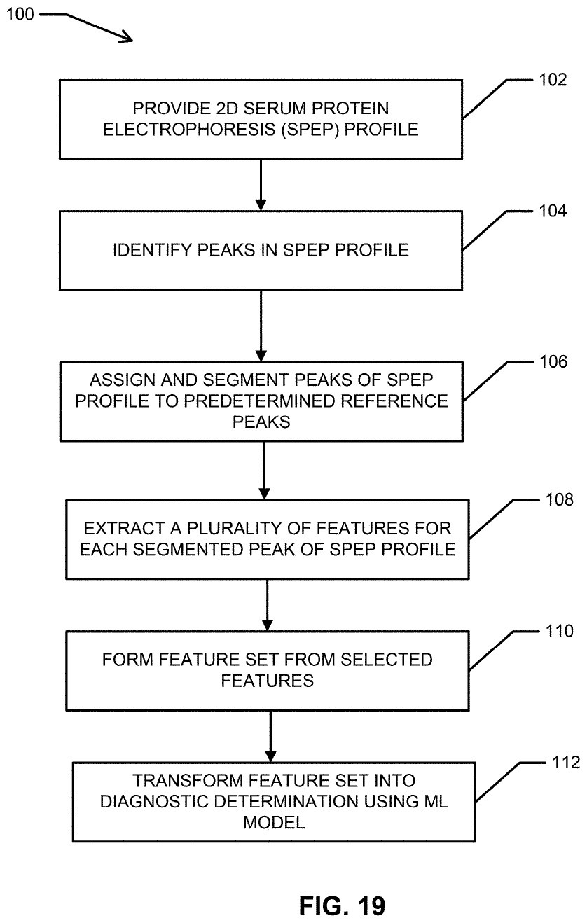

FIG. 19 is a flow chart illustrating the method of automatically generating diagnostic comments for protein capillary electrophoresis data in accordance with one aspect of the disclosure.

There are shown in the drawings arrangements that are presently discussed, it being understood, however, that the present embodiments are not limited to the precise arrangements and are instrumentalities shown. While multiple embodiments are disclosed, still other embodiments of the present disclosure will become apparent to those skilled in the art from the following detailed description, which shows and describes illustrative aspects of the disclosure. As will be realized, the invention is capable of modifications in various aspects, all without departing from the spirit and scope of the present disclosure. Accordingly, the drawings and detailed description are to be regarded as illustrative in nature and not restrictive.

### DETAILED DESCRIPTION

In various aspects, a computer-implemented method was developed to accurately and automatically label two-dimensional serum protein electrophoresis profiles (SPEPs) with appropriate clinical interpretations. The disclosed method makes use of a machine-learning model to automatically review SPEP results and generate diagnostic comments. In various aspects, the disclosed method includes automatically extracting and annotating features of interest (“peaks” and “regions”) in protein electrophoresis data and producing a correct interpretive comment for the SPEP results based on these extracted features using a pre-trained machine learning model as described in additional detail herein.

A flow chart illustrating the steps of the method for automatically generating diagnostic comments for protein capillary electrophoresis data is provided at FIG. 19. The method 100 includes providing protein capillary electrophoresis data at 102. In various aspects, any suitable protein capillary electrophoresis data may be provided at 102 without limitation. In various aspects, the protein capillary electrophoresis data comprises at least one 2-dimensional protein capillary electrophoresis profile comprising a plurality of intensity values and corresponding times. In one aspect, the protein capillary electrophoresis data is at least one 2D serum protein electrophoresis (SPEP) profile. A non-limiting example of a SPEP data profile is provided at FIG. 7A.

Referring again to FIG. 7A, the method further includes identifying and assigning peaks within the 2D SPEP profile at 104. By way of non-limiting example, the raw intensities comprising a vector of 300 abundance values (see FIG. 7A) are smoothed using local polynomial regression (loess) to produce a continuous smoothed function as illustrated in FIG. 7B. Candidate peaks are identified by calculating the first derivative at each point and selecting all positions where there is a sign change in the first derivative, with the first derivative at both points to the immediate left of the candidate peak being positive and the first derivative at both points to the immediate right of the candidate peak being negative. Candidate peaks are filtered to remove positions within the first 60 (1-60) or final 10 (291-300) points of the trace. Up to eight peaks are identified for each sample, taking the eight candidate peaks with the lowest second derivatives. In other aspects, the number of peaks selected for subsequent analysis as described below may be at least 4, 6, 8, 10, 12, or more.

In various aspects, the method further includes assigning the candidate peaks to predetermined reference peaks at 106, In various aspects, the reference peaks are indicative of individual proteins within the sample to be detected using serum protein electrophoresis. In one aspect, the predetermined reference peaks include peaks indicative of the serum proteins albumin, alpha-1, alpha-2, beta-1, beta-2, and gamma. In various aspects, the x-coordinates of reference peaks (albumin, alpha-1, alpha-2, beta-1, beta-2, and gamma) are defined by calculating the average profile over all traces in the dataset, identifying the position of the six highest local maxima, and assigning reference peak labels in left-to-right order (albumin, alpha-1, alpha-2, beta-1, beta-2, and gamma).

To assign candidate peaks to reference peaks for an individual trace, all possible assignments of candidate peaks to reference peaks are scored using the following metric:

$$s = {\sum }_{i} \frac{{y}_{i}}{{( 1 + \vert {x}_{i} - {x}_{r} \vert )}^{1 / 2}}$$

where |xᵢ−xᵣ| is the horizontal distance from candidate peak ‘i’ to the assigned reference peak and yᵢ is the trace height at position xᵢ. The configuration yielding the highest score s is then used to define which candidate peaks correspond to specific reference peaks (see FIG. 7C) and which peaks are anomalous (up to two, if present). Anomalous peaks (see FIG. 7E) are assigned to the beta-2 or gamma regions based on proximity (beta-2 peak 2 and/or gamma peak 2). Finally, to segment the trace into regions, regions boundaries are defined as the trace positions with the lowest heights between adjacent pairs of reference peaks (see FIGS. 7D and 7E).

Referring again to FIG. 19, for each segmented region, a plurality of features are extracted at 108. In various aspects, the features correspond to mathematical expressions capturing one or more characteristics of each peak that are typically considered by a practitioner when making a diagnostic determination using a manual evaluation of an SPEP profile including, but not limited to, peak location, curvature, symmetry, smoothness, separation from other peaks, and any other suitable characteristic without limitation. In various aspects, the plurality of features for each assigned peak/region includes at least one of a peak feature, a region feature, a miscellaneous feature, and any combination thereof.

Non-limiting examples of suitable peak features include peak curvature, peak first derivative (left), peak first derivative (right), peak second derivative (left), peak second derivative (right), peak x-coordinate, and peak y-coordinate. Peak curvature, as used herein, refers to the inverse of the radius of the circle defined by the peak and the points on its immediate left and right. Peak first derivative (left), as used herein, refers to the mean first derivative of the three points to the immediate left of the peak. Peak first derivative (right), as used herein, refers to the mean first derivative of the three points to the immediate right of the peak. Peak second derivative (left), as used herein, refers to the mean second derivative of the three points to the immediate left of the peak. Peak second derivative (right), as used herein, refers to the mean second derivative of the three points to the immediate right of the peak. Peak x-coordinate, as used herein, refers to the horizontal location of the peak. Peak y-coordinate, as used herein, refers to the peak height.

Non-limiting examples of suitable region features include Area under the curve, Center of mass, Peak angle, Polynomial fit (degree 2), Polynomial fit (degree 4), Polynomial fit (degree 4), Polynomial fit (degree 8), Polynomial fit (degree 10), Region curvature, Region second derivative (minimum), Skew, Slope (left), Slope (right), Smoothness 1, and Smoothness 2. The area under the curve, as used herein, refers to the sum of y-values in the region. Center of mass, as used herein, refers to the sum of the products of each x-coordinate and y-coordinate in the region divided by the area under the curve. Peak angle, as used herein, refers to the angle (in degrees) formed by the peak and the two points defining the adjacent region boundaries. Polynomial fit (degree 2), as used herein, refers to the root-mean-square error of second-degree polynomial function fit over the region, scaled to the maximum y-value in the region. Polynomial fit (degree 4), as used herein, refers to the root-mean-square error of fourth-degree polynomial function fit over the region, scaled to the maximum y-value in the region. Polynomial fit (degree 6), as used herein, refers to the root-mean-square error of sixth-degree polynomial function fit over the region, scaled to the maximum y-value in the region. Polynomial fit (degree 8), as used herein, refers to the root-mean-square error of eighth-degree polynomial function fit over the region, scaled to the maximum y-value in the region. Polynomial fit (degree 10), as used herein, refers to the root-mean-square error of tenth-degree polynomial function fit over the region, scaled to the maximum y-value in the region. Region curvature, as used herein, refers to the average curvature over the region scaled to the maximum y-value in the region. Region second derivative (minimum), as used herein, refers to the minimum second derivative scaled to the maximum y-value in the region. Skew, as used herein, refers to the absolute difference between the center of mass and peak x-coordinate. Slope (left), as used herein, refers to the slope of the line connecting the point defining the left region boundary to the region peak. Slope (right), as used herein, refers to the slope of the line connecting the point defining the right region boundary to the region peak. Smoothness 1, as used herein, refers to the number of sign changes in the first derivative over the region. Smoothness 2, as used herein, refers to the average of the second derivative squared scaled to the maximum y-value in the region.

Non-limiting examples of suitable miscellaneous features include Inter-region angle (beta-1, beta-2), Inter-region angle (beta-2, gamma), and Inter-region angle (beta-2, gamma, vertical). Inter-region angle (beta-1, beta-2), as used herein, refers to the angle defined by the beta-1 and beta-2 peaks and the intervening region boundary. Inter-region angle (beta-2, gamma), as used herein, refers to the angle defined by the beta-2 and gamma peaks and the intervening region boundary. Inter-region angle (beta-2, gamma, vertical), as used herein, refers to the upper angle defined by the intersection of a vertical line drawn through the beta-2:gamma boundary point and a line drawn from the beta-2:gamma boundary point to the gamma peak.

Referring again to FIG. 19, the method further includes selecting at least a portion of the extracted features at 110 to form a feature set for subsequent analysis using a machine learning model. Any portion of all extracted featured from the features extracted for all peaks/regions at 108 may be selected for inclusion in the feature set ranging from at least 40, at least 50, at least 60, at least 70, at least 80, at least 90, at least 100, at least 120, at least 130, at least 140, at least 160, and at least 200 features. In one exemplary aspect (see FIG. 8), the feature set includes 107 features, including the area under the curve, peak x-coordinate, peak y-coordinate for Albumin and alpha-1 (n=2×3=6); area under the curve, peak angle, peak curvature, peak first derivative (left), peak first derivative (right), peak second derivative (left), peak second derivative (right), peak x-coordinate, peak y-coordinate, polynomial fit (degree 2), polynomial fit (degree 4), polynomial fit (degree 6), polynomial fit (degree 8), polynomial fit (degree 10), region curvature, region second derivative (minimum), skew, slope (left), slope (right), smoothness 1, smoothness 2 for Alpha-2, beta-1, beta-2, gamma (n=4×21=84); peak curvature, peak first derivative (left), peak first derivative (right), peak second derivative (left), peak second derivative (right), peak x-coordinate, peak y-coordinate for Beta-2 peak 2, gamma peak 2 (n=2×7=14), as well as inter-region angle (beta-1, beta-2), inter-region angle (beta-2, gamma), and inter-region angle (beta-2, gamma, vertical).

In various aspects, the method further includes transforming the feature set into a clinical comment using a machine learning model at 112. Any suitable machine learning model may be used without limitation including, but not limited to the ML models described in the examples below. In some aspects, the feature set includes all extracted features and the ML model includes a subset of the features set for analysis as described below. In other aspects, the ML model makes use of all features provided in the feature set.

In various aspects, the ML model output is a list of possible classifications (clinical comments) and confidences associated with each possible comment, as shown in FIGS. 15A, 15B, 15C, and 15D and as described in the example below. In some aspects, a determination is made based on confidence thresholds including, but not limited to, selecting the clinical comment category with max confidence, or any other suitable criterion.

In some aspects, a feature extraction routine for serum protein electrophoresis data works as follows: 1) first and second finite differences are used to identify local maxima (up to eight candidate peaks per case), 2) a scoring function is used to identify the optimal correspondence between candidate peaks and reference peaks (albumin, alpha-1, alpha-2, beta-1, beta-2, and gamma), allowing up to two anomalous peaks, and 3) the identified peaks are used to partition the curve into regions (albumin, alpha-1, alpha-2, beta-1, beta-2, and gamma). The following features are calculated (if necessary) and extracted for each peak: x-coordinate, y-coordinate, local curvature (3-unit window), local angle (3-unit window), leading and lagging first derivatives (mean, 5-unit window), and leading and lagging second derivatives (mean, 5-unit window). The following features are calculated for each region: area under the curve, skewness, smoothness, number of inflection points/bending energy, mean curvature, minimum of the second derivative, mean sum of squares of the second derivative, slopes of the segments connecting each region boundary (start and end) to its associated peak, angle formed by adjoining boundaries and peak (start-peak-end), angle formed by adjacent peaks through the joining boundary (peak-boundary-peak), and the root mean squared errors of polynomial fits (degree 2, 4, 6, 8, and 10). The resulting representation of the data is a vector consisting of 107 values.

This 107-value vector is then passed to pre-trained machine learning models optimized for specific tasks. For serum protein electrophoresis, a penalized regression model was trained to execute a binary classification task (normal vs. abnormal) using a manually curated dataset of 6737 clinical samples that had been interpreted as part of routine clinical care. In addition, a gradient boosting machine was trained using the same dataset to identify specific abnormalities (normal vs. abnormal restricted peak in beta-1, abnormal restricted peak in beta-2, abnormal restricted peak in gamma, possible abnormal restricted peak in beta-2, and possible abnormal restricted peak in gamma). In addition to providing class predictions, both models report predicted class probabilities (reflecting the confidence of the model prediction), which can be used to triage samples and identify difficult cases requiring further review.

The most important features used by the ML model (FIG. 16) included smoothness (β1, β2, γ), AUC (β2), and Steepness (β1, β2).

The data processing and modeling steps described above were implemented in the open-source programing language R, using the package Hmisc (v4.4-2) as well as the meta-packages tidyverse (v1.3.0) and tidymodels (v0.1.2).

As described herein, these tools constitute an advance over the current technology by automating repetitive and time-consuming manual review, reducing the amount of hands-on-time required by laboratory technologists, reducing the amount of training required of laboratory technologists, reducing the number of cases requiring medical director revision, reducing transcriptional errors, and decreasing turnaround time. In addition, these tools promote standardization. For example, the interpretation of serum protein electrophoresis is subject to inter-reviewer variability, with different human experts disagreeing on whether subtle variations in protein traces constitute normal vs. abnormal patterns. By extracting rich representations of these data and deriving robust quantitative rules to identify specific patterns, the methods disclosed herein make the interpretation of protein electrophoresis data more objective. This has the potential to deliver more consistent and higher-quality results across individual reviewers and institutions.

In various aspects, the disclosed method may be deployed as a native program within capillary electrophoresis instrument software or as a standalone application capable of interfacing with instrument software. The application would present reviewers with individual traces and model predictions for each sample and allow users to accept or override each interpretation. For all traces, the application would present the estimated class probabilities of each candidate diagnostic comment (conveying model uncertainty). For traces with high-confidence predictions (based on a user-defined threshold for acceptable accuracy), the application would automatically select the most likely diagnostic comment and flag the result as high-confidence to facilitate throughput (i.e., triage simple cases). For traces with low-confidence predictions, the application would not select a single interpretation but would highlight the most likely candidate comments and flag the sample as requiring additional investigation. FIG. 12 is a graph summarizing the accuracy of the predictions of the machine learning model as a function of probability/confidence thresholds.

In addition to distributing this application with pre-trained models and fixed diagnostic comments, it could be distributed with functionality to allow users to fine-tune models using local data and/or train custom models (using the same feature set) to accommodate user-specific labels.

In some aspects, the disclosed method may be used to monitor a patient during a treatment by analyzing SPEP samples taken at various points in the treatment. In some aspects, if a SPEP profile is categorized by the ML model as having higher confidence in a normal (NMPD, no apparent monoclonal peak) classification relative to the corresponding pre-treatment categorization, the efficacy of the treatment is indicated. Conversely, if the SPEP profile is categorized by the ML model as having lower or unchanged confidence in a normal (NMPD, no apparent monoclonal peak) classification relative to the corresponding pre-treatment categorization, low efficacy of the treatment is indicated. In various aspects, the disclosed method may be used to select, adjust, or terminate a treatment based on the efficacy of the treatment as indicated by changes in the clinical classification of SPEP data obtained using the disclosed method.

### Computing Systems and Devices

FIG. 1 depicts a simplified block diagram of a computing device for implementing the methods of analyzing the results obtained by a serum protein electrophoresis (SPEP) system as described herein. As illustrated in FIG. 1, the computing device 300 may be configured to implement at least a portion of the tasks associated with the disclosed method including, but not limited to: operating the SPEP system 310 to obtain serum protein electrophoresis data including, but not limited to, two-dimensional protein electrophoresis profiles from a serum sample. The computer system 300 may include a computing device 302. In one aspect, the computing device 302 is part of a server system 304, which also includes a database server 306. The computing device 302 is in communication with a database 308 through the database server 306. The computing device 302 is communicably coupled to the SPEP system 310 and a user computing device 330 through a network 350. The network 350 may be any network that allows local area or wide area communication between the devices. For example, the network 350 may allow communicative coupling to the Internet through at least one of many interfaces including, but not limited to, at least one of a network, such as the Internet, a local area network (LAN), a wide area network (WAN), an integrated services digital network (ISDN), a dial-up-connection, a digital subscriber line (DSL), a cellular phone connection, and a cable modem. The user computing device 330 may be any device capable of accessing the Internet including, but not limited to, a desktop computer, a laptop computer, a personal digital assistant (PDA), a cellular phone, a smartphone, a tablet, a phablet, wearable electronics, smart watch, or other web-based connectable equipment or mobile devices.

In other aspects, the computing device 302 is configured to perform a plurality of tasks associated with obtaining SPEP analysis results. FIG. 2 depicts a component configuration 400 of computing device 402, which includes database 410 along with other related computing components. In some aspects, computing device 402 is similar to computing device 302 (shown in FIG. 1). A user 404 may access components of computing device 402. In some aspects, database 410 is similar to database 308 (shown in FIG. 1).

In one aspect, database 410 includes SPEP data 418, algorithm data 420. Non-limiting examples of suitable SPEP data include the two-dimensional protein electrophoresis profiles obtained by the SPEP system 310, the feature sets obtained by the feature extraction routines as described above, and the automatically-generated interpretative comments obtained using the pre-trained machine learning models as described above. Non-limiting examples of suitable algorithm data 420 include any values of parameters defining the feature extraction algorithms described above. Non-limiting examples of suitable ML data 416 include any of the parameters describing the machine learning models used to generate the interpretive comments for the two-dimensional protein electrophoresis profiles based on the feature sets as described above.

Computing device 402 also includes a number of components that perform specific tasks. In the exemplary aspect, the computing device 402 includes a data storage device 430, ML component 440, SPEP component 450, feature extraction component 470, and communication component 460. The data storage device 430 is configured to store data received or generated by computing device 402, such as any of the data stored in database 410 or any outputs of processes implemented by any component of computing device 402. SPEP component 450 is configured to operate, or produce signals configured to operate, a SPEP device to obtain SPEP data. Feature extraction component 470 is configured to generate a feature set based on the SPEP data as described above. ML component is configured to generate appropriate diagnostic comments based on the feature set.

The communication component 460 is configured to enable communications between computing device 402 and other devices (e.g. user computing device 330 and IMRT system 310, shown in FIG. 1) over a network, such as the network 350 (shown in FIG. 1), or a plurality of network connections using predefined network protocols such as TCP/IP (Transmission Control Protocol/Internet Protocol).

FIG. 3 depicts a configuration of a remote or user computing device 502, such as user computing device 330 (shown in FIG. 1). Computing device 502 may include a processor 505 for executing instructions. In some aspects, executable instructions may be stored in a memory area 510. Processor 505 may include one or more processing units (e.g., in a multi-core configuration). The memory area 510 may be any device allowing information such as executable instructions and/or other data to be stored and retrieved. Memory area 510 may include one or more computer-readable media.

Computing device 502 may also include at least one media output component 515 for presenting information to a user 501. Media output component 515 may be any component capable of conveying information to user 501. In some aspects, media output component 515 may include an output adapter, such as a video adapter and/or an audio adapter. An output adapter may be operatively coupled to processor 505 and operatively coupleable to an output device such as a display device (e.g., a liquid crystal display (LCD), an organic light-emitting diode (OLED) display, cathode ray tube (CRT), or “electronic ink” display) or an audio output device (e.g., a speaker or headphones). In some aspects, media output component 515 may be configured to present an interactive user interface (e.g., a web browser or client application) to user 501.

In some aspects, computing device 502 may include an input device 520 for receiving input from user 501. Input device 520 may include, for example, a keyboard, a pointing device, a mouse, a stylus, a touch-sensitive panel (e.g., a touchpad or a touch screen), a camera, a gyroscope, an accelerometer, a position detector, and/or an audio input device. A single component such as a touch screen may function as both an output device of media output component 515 and input device 520.

Computing device 502 may also include a communication interface 525, which may be communicatively coupleable to a remote device. Communication interface 525 may include, for example, a wired or wireless network adapter or a wireless data transceiver for use with a mobile phone network (e.g., Global System for Mobile communications (GSM), 3G, 4G or Bluetooth) or other mobile data network (e.g., Worldwide Interoperability for Microwave Access (WIMAX)).

Stored in memory area 510 are, for example, computer-readable instructions for providing a user interface to user 501 via media output component 515 and, optionally, receiving and processing input from input device 520. A user interface may include, among other possibilities, a web browser and client application. Web browsers enable users 501 to display and interact with media and other information typically embedded on a web page or a website from a web server. A client application allows users 501 to interact with a server application associated with, for example, a vendor or business.

FIG. 4 illustrates an example configuration of a server system 602. Server system 602 may include, but is not limited to, database server 306 and computing device 302 (both shown in FIG. 1). In some aspects, server system 602 is similar to server system 304 (shown in FIG. 1). Server system 602 may include a processor 605 for executing instructions. Instructions may be stored in a memory area 625, for example. Processor 605 may include one or more processing units (e.g., in a multi-core configuration).

Processor 605 may be operatively coupled to a communication interface 615 such that server system 602 may be capable of communicating with a remote device such as user computing device 330 (shown in FIG. 1) or another server system 602. For example, communication interface 615 may receive requests from a user computing device 330 via a network 350 (shown in FIG. 1).

Processor 605 may also be operatively coupled to a storage device 625. Storage device 625 may be any computer-operated hardware suitable for storing and/or retrieving data. In some aspects, storage device 625 may be integrated with server system 602. For example, server system 602 may include one or more hard disk drives as storage device 625. In other aspects, storage device 625 may be external to server system 602 and may be accessed by a plurality of server systems 602. For example, storage device 625 may include multiple storage units such as hard disks or solid-state disks in a redundant array of inexpensive disks (RAID) configuration. Storage device 625 may include a storage area network (SAN) and/or a network attached storage (NAS) system.

In some aspects, processor 605 may be operatively coupled to storage device 625 via a storage interface 620. Storage interface 620 may be any component capable of providing processor 605 with access to storage device 625. Storage interface 620 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing processor 605 with access to storage device 625.

Memory areas 510 (shown in FIG. 3) and 610 may include, but are not limited to, random access memory (RAM) such as dynamic RAM (DRAM) or static RAM (SRAM), read-only memory (ROM), erasable programmable read-only memory (EPROM), electrically erasable programmable read-only memory (EEPROM), and non-volatile RAM (NVRAM). The above memory types are examples only and are thus not limiting as to the types of memory usable for the storage of a computer program.

The computer systems and computer-implemented methods discussed herein may include additional, less, or alternate actions and/or functionalities, including those discussed elsewhere herein. The computer systems may include or be implemented via computer-executable instructions stored on non-transitory computer-readable media. The methods may be implemented via one or more local or remote processors, transceivers, servers, and/or sensors (such as processors, transceivers, servers, and/or sensors mounted on vehicle or mobile devices, or associated with smart infrastructure or remote servers), and/or via computer-executable instructions stored on non-transitory computer-readable media or medium.

In some aspects, a computing device is configured to implement machine learning, such that the computing device “learns” to analyze, organize, and/or process data without being explicitly programmed. Machine learning may be implemented through machine learning (ML) methods and algorithms. In one aspect, a machine learning (ML) module is configured to implement ML methods and algorithms. In some aspects, ML methods and algorithms are applied to data inputs and generate machine learning (ML) outputs. Data inputs may further include: sensor data, image data, video data, telematics data, authentication data, authorization data, security data, mobile device data, geolocation information, transaction data, personal identification data, financial data, usage data, weather pattern data, “big data” sets, and/or user preference data. In some aspects, data inputs may include certain ML outputs.

In some aspects, at least one of a plurality of ML methods and algorithms may be applied, which may include but are not limited to: linear or logistic regression, instance-based algorithms, regularization algorithms, decision trees, Bayesian networks, cluster analysis, association rule learning, artificial neural networks, deep learning, dimensionality reduction, and support vector machines. In various aspects, the implemented ML methods and algorithms are directed toward at least one of a plurality of categorizations of machine learning, such as supervised learning, unsupervised learning, and reinforcement learning. In some aspects, different machine learning models may be used to generate different portions of the desired results including, but not limited to, penalized regression models for binary classification tasks and gradient boosting machines to identify specific abnormalities.

In one aspect, ML methods and algorithms are directed toward supervised learning, which involves identifying patterns in existing data to make predictions about subsequently received data. Specifically, ML methods and algorithms directed toward supervised learning are “trained” through training data, which includes example inputs and associated example outputs. Based on the training data, the ML methods and algorithms may generate a predictive function that maps outputs to inputs and utilize the predictive function to generate ML outputs based on data inputs. The example inputs and example outputs of the training data may include any of the data inputs or ML outputs described above.

In another aspect, ML methods and algorithms are directed toward unsupervised learning, which involves finding meaningful relationships in unorganized data. Unlike supervised learning, unsupervised learning does not involve user-initiated training based on example inputs with associated outputs. Rather, in unsupervised learning, unlabeled data, which may be any combination of data inputs and/or ML outputs as described above, is organized according to an algorithm-determined relationship.

In yet another aspect, ML methods and algorithms are directed toward reinforcement learning, which involves optimizing outputs based on feedback from a reward signal. Specifically, ML methods and algorithms directed toward reinforcement learning may receive a user-defined reward signal definition, receive a data input, utilize a decision-making model to generate an ML output based on the data input, receive a reward signal based on the reward signal definition and the ML output, and alter the decision-making model so as to receive a stronger reward signal for subsequently generated ML outputs. The reward signal definition may be based on any of the data inputs or ML outputs described above. In one aspect, an ML module implements reinforcement learning in a user recommendation application. The ML module may utilize a decision-making model to generate a ranked list of options based on user information received from the user and may further receive selection data based on a user selection of one of the ranked options. A reward signal may be generated based on comparing the selection data to the ranking of the selected option. The ML module may update the decision-making model such that subsequently generated rankings more accurately predict a user selection.

As will be appreciated based upon the foregoing specification, the above-described aspects of the disclosure may be implemented using computer programming or engineering techniques including computer software, firmware, hardware, or any combination or subset thereof. Any such resulting program, having computer-readable code means, may be embodied or provided within one or more computer-readable media, thereby making a computer program product, i.e., an article of manufacture, according to the discussed aspects of the disclosure. The computer-readable media may be, for example, but is not limited to, a fixed (hard) drive, diskette, optical disk, magnetic tape, semiconductor memory such as read-only memory (ROM), and/or any transmitting/receiving medium, such as the Internet or other communication network or link. The article of manufacture containing the computer code may be made and/or used by executing the code directly from one medium, by copying the code from one medium to another medium, or by transmitting the code over a network.

These computer programs (also known as programs, software, software applications, “apps”, or code) include machine instructions for a programmable processor, and can be implemented in a high-level procedural and/or object-oriented programming language, and/or in assembly/machine language. As used herein, the terms “machine-readable medium” “computer-readable medium” refers to any computer program product, apparatus, and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The “machine-readable medium” and “computer-readable medium,” however, do not include transitory signals. The term “machine-readable signal” refers to any signal used to provide machine instructions and/or data to a programmable processor.

As used herein, a processor may include any programmable system including systems using micro-controllers, reduced instruction set circuits (RISC), application specific integrated circuits (ASICs), logic circuits, and any other circuit or processor capable of executing the functions described herein. The above examples are examples only, and are thus not intended to limit in any way the definition and/or meaning of the term “processor.”

As used herein, the terms “software” and “firmware” are interchangeable and include any computer program stored in memory for execution by a processor, including RAM memory, ROM memory, EPROM memory, EEPROM memory, and non-volatile RAM (NVRAM) memory. The above memory types are examples only and are thus not limiting as to the types of memory usable for the storage of a computer program.

In one aspect, a computer program is provided, and the program is embodied on a computer-readable medium. In one aspect, the system is executed on a single computer system, without requiring a connection to a server computer. In a further aspect, the system is being run in a Windows® environment (Windows is a registered trademark of Microsoft Corporation, Redmond, Wash.). In yet another aspect, the system is run on a mainframe environment and a UNIX® server environment (UNIX is a registered trademark of X/Open Company Limited located in Reading, Berkshire, United Kingdom). The application is flexible and designed to run in various different environments without compromising any major functionality.

In some aspects, the system includes multiple components distributed among a plurality of computing devices. One or more components may be in the form of computer-executable instructions embodied in a computer-readable medium. The systems and processes are not limited to the specific aspects described herein. In addition, components of each system and each process can be practiced independently and separate from other components and processes described herein. Each component and process can also be used in combination with other assembly packages and processes. The present aspects may enhance the functionality and functioning of computers and/or computer systems.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

Any publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following examples illustrate various aspects of the disclosure.

### Example 1: Automated SPEP Interpretation

To develop and validate the disclosed method of feature extraction and machine learning models for automated interpretation of serum protein electrophoresis (SPEP) data, the following experiments were conducted.

The workflow of the development of the disclosed method is provided in the block diagram shown in FIG. 6 and FIG. 14. As described below, a training dataset comprising SPEP results and associated diagnostic comments were subjected to feature extraction, and various combinations of the extracted features evaluated in a model fitting process to produce a final machine learning model. A test dataset comprising SPEP results and associated diagnostic comments were similarly subjected to feature extraction, and a final machine learning model was used to predict a clinical diagnosis based on the selected combination of extracted features as determined by the model fitting process.

A SPEP dataset containing SPEP results and diagnostic comments (n=6737) was divided into a training dataset (80% of the dataset) and a test dataset (20% of the dataset) used to develop and validate the machine learning models. Table 1 below is a summary of the SPEP dataset divided into diagnostic comment groups.

TABLE 1
SPEP Dataset Characterization

| Label (abbreviation) | Count (n) | Proportion (%) |
|---|---|---|
| No apparent monoclonal peak (NMPD) | 2279 | 34 |
| Abnormal restricted peak in gamma region | 2241 | 33 |
| (ARP-G) | | |
| Possible abnormal restricted peak in gamma | 1494 | 22 |
| region (PARP-G) | | |
| Possible abnormal restricted peak in beta-2 | 255 | 4 |
| region (PARP-B2) | | |
| Abnormal restricted peak in beta-2 region | 370 | 6 |
| (ARP-B2) | | |
| Abnormal restricted peak in beta-1 region | 61 | 1 |
| (ARP-B1) | | |
| Abnormal restricted peak in alpha-2 region | 19 | <1 |
| (ARP-A2) | | |
| TOTAL | 6719 | 100 |

FIGS. 5A, 5B, 5C, and 5D are exemplary SPEP profiles from the dataset. FIG. 5A is an SPEP profile labeled as normal. FIG. 5B and FIG. 5C are SPEP profiles labeled as an abnormal restricted peak in the gamma region, although the magnitudes and shapes of the abnormal peaks are different. FIG. 5D is an SPEP profile labeled as a possible abnormal restricted peak in the gamma region.

Each SPEP result was subjected to feature extraction to transform the SPEP profile into a plurality of features indicative of the size and shape of the various peaks within the SPEP profile. Initially, a SPEP trace (FIG. 7A) was analyzed using first and second finite differences to identify local maxima, and candidate peaks were then assigned to albumin, alpha-1, alpha-2, beta-1, beta-2, and gamma (FIG. 7B). The SPEP trace was then segmented into regions around each candidate peak (FIG. 7C). For each peak, a plurality of peak features was calculated, including x-coordinate, y-coordinate, local curvature (3-unit window), local angle (3-unit window), leading and lagging first derivatives (mean, 5-unit window), and leading and lagging second derivatives (mean, 5-unit window). For each segmented region, a plurality of area features was extracted, including area under the curve, skew, number of inflection points, mean curvature, minimum of the second derivative, mean sum of squares of the second derivative, slopes of the segments connecting each region boundary to its associated peak, angle formed by adjacent peaks through the joining boundary, and the root mean squared errors of polynomial fits (degree 2, 4, 6, 8, and 10). A subset of the peak features and area features were assembled into a feature set, as illustrated in FIG. 8.

Four different machine learning models were trained and evaluated using the feature sets derived from the training dataset: KNN, elastic net regression, random forests, and gradient boosting machine. The hyperparameters of each machine learning model were tuned using repeated cross-validation (5×5), and final hyperparameters were selected based on average performance over cross-validation folds.

The machine learning models were validated by subjecting the test dataset to feature extraction, transforming the feature sets to predicted diagnostic comments, and calculating performance metrics based on these results. FIGS. 9A and 9B are graphs comparing ROC (FIG. 9A) and precision/recall (FIG. 9B) of the four machine learning models for a binary classification task (normal/abnormal). FIG. 10 is the ROC curve for the elastic net model with several operating points labeled.

Performance metrics were calculated on the test set and are summarized in Tables 2 and 3. Briefly, the highest point estimates for the area under the receiver operating characteristic curve (AUC-ROC) and area under the precision-recall (AUC-PR) curve for binary classification (normal vs. abnormal) were achieved with penalized logistic regression (0.985 and 0.993, respectively). The highest point estimates for AUC-ROC and AUC-PR for multiclass classification (predicting the specific diagnostic comment) were achieved with gradient boosted trees (0.978 and 0.895, respectively). While these models achieved the highest point estimates for these tasks, bootstrap confidence intervals indicate that the performance for penalized logistic regression, random forests, and gradient boosted trees were comparable.

FIGS. 9A and 9B are graphs comparing ROC (FIG. 9A) and precision/recall (FIG. 9B) of the four machine learning models for a binary classification task (normal/abnormal).

TABLE 2
ML Model Performance

| Classification | | AUC-ROC | AUC-PR |
|---|---|---|---|
| Task | Model | (95% CI) | (95% CI) |
| Binary | K-Nearest Neighbors | 0.948 | 0.976 |
| | (KNN) | (0.937,0.958) | (0.970,0.981) |
| | Penalized Logistic | 0.985 | 0.993 |
| | Regression | (0.980,0.990) | (0.990,0.995) |
| | Random Forest | 0.981 | 0.991 |
| | | (0.976,0.986) | (0.987,0.993) |
| | Gradient Boosted | 0.985 | 0.992 |
| | Tree | (0.980,0.989) | (0.990,0.995) |
| Multiclass | K-Nearest Neighbors | 0.938 | 0.799 |
| | (KNN) | (0.913,0.952) | (0.758,0.835) |
| | Penalized Logistic | 0.972 | 0.847 |
| | Regression | (0.957,0.978) | (0.809,0.880) |
| | Random Forest | 0.974 | 0.867 |
| | | (0.961,0.982) | (0.829,0.905) |
| | Gradient Boosted | 0.978 | 0.895 |
| | Tree | (0.966,0.984) | (0.868,0.923) |

TABLE 3
ML Model Performance (Multiclass Classification)

| Model | Accuracy |
|---|---|
| K-Nearest Neighbors (KNN) | 0.75 |
| Random Forest | 0.88 |
| Penalized Logistic Regression | 0.86 |
| Gradient Boosted Tree | 0.88 |

To place these performance metrics in the context of current practice, an experiment was performed to characterize the variability of human serum protein electrophoresis interpretation and to compare the performance of the model to human experts. Briefly, a random sample of 100 traces was provided to five human experts, who were asked to interpret each according to their standard practice. For binary classification, the median Cohen's kappa between all pairs of reviewers was 0.70 (range 0.31-0.80). After a washout period of 4 weeks, reviewers were asked to interpret the same 100 traces again, at which time the median pairwise Cohen's kappa between reviewers was 0.60 (range 0.32-0.79). Comparing individual reviewers between time points, the median intra-reviewer Cohen's kappa was 0.70 (range 0.61-0.87). These results are consistent with significant inter- and intra-reviewer variability with respect to interpreting serum protein electrophoresis data. In contrast, the models described above are deterministic and yield the same interpretation for an individual trace each time it is analyzed.

To directly compare the performance of human reviewers to that of the model, the consensus labels assigned by the human reviewers (aggregating responses from both time points) were determined for each trace to use as a reference. The median Cohen's kappa comparing reviewer interpretations to the consensus label was 0.85 (range 0.36-0.93) during the reviewers' first evaluation and 0.78 (range 0.52-0.96) during their second (FIG. 17). In comparison, the Cohen's kappa between the model predictions and the consensus labels was 0.89. These data suggest that the model performs comparably human experts.

Finally, to determine if providing the model predictions to reviewers could help standardize their interpretation, reviewers were provided with the same set of 100 traces a third time, except this time the traces included the model predictions (estimated probability for each class). Whereas the median pairwise Cohen's kappa between reviewers was 0.64 (range 0.31-0.80) during the first two rounds of evaluation, the median pairwise Cohen's kappa between reviewers increased to 0.77 (range 0.56-0.91) when they were provided the model predictions (FIG. 18), and this increase was significant (p<0.05).

The above non-limiting example is provided to further illustrate the present disclosure. It should be appreciated by those of skill in the art that the techniques disclosed in the examples represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

1. A computer-implemented method for automatically generating diagnostic comments for protein capillary electrophoresis data obtained for a subject, the method comprising:
a. providing at least one two-dimensional serum protein electrophoresis (SPEP) profile comprising a plurality of measured abundances and corresponding times;
b. extracting, using the computing device, a feature set from the SPEP profile, the feature vector comprising at least one feature of the at least one two-dimensional protein electrophoresis profile, wherein the at least one feature comprises at least one identified peak, at least one region corresponding to each identified beak, at least one peak feature associated with each identified peak, and at least one region feature associated with each region; and
c. transforming, using a machine-learning model implemented on the computing device, the feature vector into the diagnostic comments and corresponding confidences of each diagnostic comment.

2. The method of claim 1, wherein the peak feature comprises at least one of an x-coordinate, a y-coordinate, a local curvature (3-unit window), a local angle (3-unit window), a leading and a lagging first derivative (mean, 5-unit window), a leading and a lagging second derivative (mean, 5-unit window), and any combination thereof.

3. The method of claim 2, wherein the at least one region feature comprises at least one of an area under the curve, a skew, a number of inflection points, a mean curvature, a minimum of the second derivative, a mean sum of squares of the second derivative, at least one slope of a segment connecting each region boundary to its associated peak, an angle formed by adjacent peaks through a joining boundary, at least one root mean squared errors of polynomial fit (degree 2, 4, 6, 8, and 10) and any combination thereof.

4. The method of claim 3, wherein extracting the feature set further comprises determining, using the computing device, a plurality of candidate peaks, selecting a portion of the candidate peaks with lowest second derivatives.

5. The method of claim 4, wherein extracting the feature set further comprises assigning, using the computing device, each candidate peak of the portion to a corresponding reference peak, wherein each reference peak is a known serum protein selected from albumin, alpha-1, alpha-2, beta-1, beta-2, and gamma.

6. The method of claim 5, wherein assigning each candidate peak further comprises assigning one or two additional candidate peaks to secondary peaks comprising secondary beta-2 or secondary gamma.

7. The method of claim 1, wherein the machine learning model comprises one of KNN, elastic net regression, random forests, and gradient boosting machine.
